(19) United States
(12) Patent Application Publication
(10) Pub. No.: US 2025/0005695 A1
(43) Pub. Date: Jan. 2, 2025

(54) CLOUD-BASED FORMULATION AND DELIVERY OF INDIVIDUAL LEVEL HOUSING-BASED SOCIOECONOMIC STATUS (HOUSES) INDEX

(71) Applicant: Mayo Foundation for Medical Education and Research, Rochester, MN (US)

(72) Inventors: Chung I. Wi, Rochester, MN (US); Young J. Juhn, Rochester, MN (US); Euijung Ryu, Rochester, MN (US); Timothy Tschampel, Carlsbad, CA (US)

(21) Appl. No.: 18/710,122

(22) PCT Filed: Nov. 15, 2022
(86) PCT No.: PCT/US2022/079888

§ 371 Date: May 14, 2024

Related U.S. Application Data
(60) Provisional application No. 63/279,616, filed on Nov. 15, 2021.

(51) Int. Cl. G06Q 50/22; G06F 21/62

(52) U.S. Cl. CPC G06Q 50/22; G06F 21/6254

(57) ABSTRACT

Housing-based socioeconomic status (“HOUSES”) index scores as an individual-level socioeconomic status (“SES”) measure are formulated and managed using a secure cloud-based interface that maintains data privacy for individuals. The cloud-based environment enables a scalable solution for generating and managing HOUSES index data by enabling access to publicly available real property data used when formulating a HOUSES index score. The cloud-based environment provides a reproducible, agile, and scalable algorithm deployment enabling the generation and management of de-identified HOUSES index data.

### CROSS-REFERENCE TO RELATED APPLICATIONS

This application claims the benefit of U.S. Provisional Patent Application Ser. No. 63/279,616, filed on Nov. 15, 2021, and entitled “Cloud-Based Formulation and Delivery of Individual Level Housing-Based Socioeconomic Status (HOUSES) Index,” which is herein incorporated by reference in its entirety.

### STATEMENT REGARDING FEDERALLY SPONSORED RESEARCH

This invention was made with government support under HD051902 and AG065639 awarded by the National Institutes of Health. The government has certain rights in the invention.

### BACKGROUND

Despite the significant role of socioeconomic status (“SES”) in a broad range of health outcomes, care quality and behavioral risk factors through health care access, health literacy and even biological pathways, the absence of individual-level SES measures in commonly used large datasets has been a major impediment to assessing and addressing the impact of SES in clinical care and research. The use of SES measures to better interpret patient health outcomes has been limited due to the lack of individual-level data. Zip code or Census geographical unit-based aggregate measures can be used, but they are known to have significant misclassification bias.

### SUMMARY OF THE DISCLOSURE

The present disclosure addresses the aforementioned drawbacks by providing a method for generating a housing-based socioeconomic status (HOUSES) index scores for an individual. A request order is received at a server by a client, where the request order includes address data for an individual including a housing unit address for the individual. Real property data are retrieved from a real property database using the server and the address data to query the real property database. The real property data include at least a number of bedrooms of the housing unit, a number of bathrooms of the housing unit, the square footage of the housing unit, and the estimated building value of the housing unit. HOUSES index scores are generated with the server based on the real property data, and the HOUSES index scores are stored on the server.

It is another aspect of the present disclosure to provide a method for quantifying artificial intelligence (AI) model bias by an individual-level socioeconomic status (SES). The method includes accessing, with a computer system, HOUSES index scores for individuals in a study cohort, where the HOUSES index scores are generated based on real property data including at least number of bedrooms, number of bathrooms, square footage of a housing unit for each individual, and estimated building value of each housing unit. A fairness metric is computed based on the HOUSES index scores using the computer system, and AI model bias by SES in the study cohort is quantified based on the fairness metric.

The foregoing and other aspects and advantages of the present disclosure will appear from the following description. In the description, reference is made to the accompanying drawings that form a part hereof, and in which there is shown by way of illustration a preferred embodiment. This embodiment does not necessarily represent the full scope of the invention, however, and reference is therefore made to the claims and herein for interpreting the scope of the invention.

### BRIEF DESCRIPTION OF THE DRAWINGS

FIG. 1 is a block diagram of an example cloud-based system for formulating, matching, and delivering housing-based socioeconomic status (“HOUSES”) index scores.

FIG. 2A is a block diagram of an example HOUSES index generating and management system.

FIG. 2B is a block diagram of example components that can implement the system of FIG. 2A.

FIG. 3 is a flowchart illustrating the steps of an example method for formulating a HOUSES index using a cloud-based request for real property data relevant to computing a HOUSES index score.

FIG. 4 is a flowchart illustrating the steps of an example method for matching a HOUSES index score for an individual using a cloud-based lookup and matching process.

FIG. 5 is a flowchart illustrating the steps of an example method for delivering a HOUSES index score for an individual using a cloud-based lookup and delivery process.

FIG. 6 is a block diagram of example computer system components that can implement the systems and methods described in the present disclosure.

### DETAILED DESCRIPTION

Described here are systems and methods for formulating and managing housing-based socioeconomic status (“HOUSES”) index data using a secure interface that maintains data privacy for individuals. The effect of socioeconomic status (“SES”) on health outcomes has been observed, but computing individual-level SES data in an efficient and secure manner remains a challenge. Most solutions that obtain individual-level SES data rely on questionnaires or interviews and are, therefore, not scalable.

The HOUSES index is an individual-level SES measure based on individual housing characteristics. Input data used to formulate the HOUSES index can include data that are publicly available from the county Assessor's office, such that calculation of the index does not require patient-reported information. The systems and methods described in the present disclosure enable a scalable solution for formulating and managing HOUSES index data that is compliant with relevant data privacy regulations (e.g., Health Insurance Portability and Accountability Act (“HIPAA”)). The HOUSES index generation and management systems and methods described in the present disclosure implement a cloud-based system where HOUSES index data can be automatically formulated and provided to users who upload relevant parcel data (e.g., address information), thereby preserving data privacy.

The HOUSES index overcomes the absence of SES measures in commonly used data sources, such as medical records or administrative datasets. The HOUSES index is a robust individual-level SES measure derived from a single factor including items of real property data, such as the number of bedrooms, number of bathrooms, square footage of the unit, and estimated building value of the unit. These housing data are publicly available and can be accessed from the county Assessor's office, or other local municipality.

For formulating a HOUSES index, addresses for study subjects at the time of index date are geocoded to link real property data of housing unit(s). Each property item corresponding to an individual's address can be standardized into a z-score and aggregated into an overall z-score (e.g., a HOUSES index) for the relevant real property data (e.g., number of bedrooms, number of bathrooms, square footage of the unit, and estimated building value of the unit), or z-scores for the relevant real property data (e.g., square footage of the unit and estimated building value of the unit (e.g., a modified HOUSES index) or estimated building value of the unit (e.g., a price-based HOUSES index)). In general, a higher HOUSES index score indicates a higher socioeconomic status. HOUSES index scores can be standardized within a county based on available real property data at a given year as real property data are ascertained and updated from the respective county Assessor's office on a regular basis. Then, the z-score of HOUSES index can be converted to HOUSES indices in quartile, decile, etc. HOUSES indices have shown strong psychometric properties and have demonstrated criterion validity such that there are moderate to good correlations with education, income, Hollingshead Index (HS), and Nakao-Treas Index (NT), among others.

HOUSES indices have been shown to predict a broad range of health outcomes for both adults and children, which are known to be inversely associated with socioeconomic status, including acute (myocardial infarction, all-cause hospitalizations, accidental falls, and critical care outcome), chronic (e.g., rheumatoid arthritis diagnosis, coronary heart disease, asthma, mood disorder, hypertension, diabetes, and vitamin D status), transplantation outcome (e.g., post-kidney transplantation graft failure), behavioral health (e.g., smoking status, obesity, advance care planning), cancer (e.g., glioma), and childhood conditions (e.g., adverse self-rated health, poorly controlled asthma per the Asthma Control Test score, invasive pneumococcal disease, and pertussis or HPV vaccine compliance, prevalence of acute and chronic conditions, low birth weight, multiple complex chronic conditions), and mortality. Of note, while the HOUSES index has moderate to good correlation with other conventional SES measures, it has been demonstrated to predict health outcomes better than other SES measures.

It is an aspect of the present disclosure to provide systems and methods for generating measures of individual-level SES, which provides an improvement over previous SES measures that are computed at the zip code level or Census geographical units-based aggregate level. These prior aggregate-level methods (e.g., Area Deprivation Index) are known to have significant misclassification bias (e.g., inaccuracies of 20-30%).

It is another aspect of the present disclosure to provide systems and methods for generating individual-level SES data (e.g., HOUSES index data) using publicly available data. For example, county and/or city assessment data used for property taxes, which are updated annually for tax purposes, can be used to formulate HOUSES index data for an individual. As a result, the HOUSES index data generated will reflect an updated individual's current SES in response to their financial or socioeconomic changes (e.g., versus educational attainment, which is static over time).

It is still another aspect of the present disclosure to provide systems and method for generating an objective measure for SES that can be assessed by certified assessors in each local county, as compared to self-reported traditional individual-level SES measures (e.g., household income or educational attainment or Census-based SES data, which are not publicly available and subject to report bias).

Advantageously, the systems and methods described in the present disclosure enable a scalable solution for generating and managing individual-level SES data (e.g., HOUSES index data), since nearly all states and counties in the US keep and update assessment data for property taxes, which are electronically available as data source for calculating individual-level SES data. A cloud-based environment is disclosed for hosting a HOUSES index computation pipeline, which provides a reproducible, agile, and scalable algorithm deployment enabling the generation and management of de-identified HOUSES index data. In an example embodiment, a composite address key is created and used to match end-user addresses at different levels of fidelity, including an exact parcel, a primary address, a street level, etc.

Additionally or alternatively, an application programming interface (“API”) service can be implemented to support the matching of end-user addresses in both single and batch modes via a privacy-preserving capability. This allows the users to utilize their address data (which is protected health information (“PHI”)) to request and match HOUSES index data while no PHI is persisted by the HOUSES index API service deployment.

Because the HOUSES index uses address information and available property data, some addresses may not match to the existing real property data (e.g., a recently built house). It is contemplated that the number of houses sharing the same 9-digit zip code (e.g., median ranges between 2 and 3) and within the same Census Block (e.g., median ranges between 6 and 14 having the same Federal Information Processing Standard (“FIPS”) code of Census Block) is relatively small, although it can differ by state. Assuming the HOUSES indices for people living in same 9-digit zip codes or Census Block are similar, missing HOUSES can be imputed using the average HOUSES of parcels sharing the same 9-digit zip code or FIPS code of Census Block instead of remaining missing or randomly assigning zero (i.e., mean of HOUSES index within a county).

Neighborhood SES, which may differ from individual-level SES (e.g., HOUSES), may have an impact on health outcomes through different mechanisms than individual-level SES. The Area Deprivation Index (“ADI”) is publicly available aggregate level measure for neighborhood environment based on 17 area-level variables (e.g., Census Block Groups). The ADI uses rankings of neighborhoods with respect to socioeconomic disadvantage (higher score, more deprived area). Given the widely recognized impact of neighborhood environment, such as ADI, on health outcomes independent of individual-level SES, the HOUSES cloud system can provide users with ADI, not as a substitute or proxy of individual-level SES measure, but as a measure of neighborhood environment via a multi-level analysis analyzing both individual and neighborhood-level measures.

Urban-rural status can be classified by people's residing address, and can be an important predictor for health outcomes and used to study and address rural health disparities. The HOUSES cloud system can provide users with rural classification defined by several methods, including Rural-Urban commuting area classification (“RUCA”) and/or Census Bureau (Urban and Rural) by using a person's street address, which is a basis of HOUSES formulation.

Distance to a health care clinic from where people live is often used as a surrogate marker for general (physical) access to health care services and timely access to critical care. For example, the distance between a patient's residence and the nearest Emergency Department can be relevant to critical outcomes requiring urgent medical or surgical interventions, such as stroke or myocardial infarction, which requires emergent intervention. Since a prerequisite for calculating the HOUSES index is to geocode an individual's residential address, this important variable can also be a byproduct of the HOUSES index and, thus, can be provided to users per request.

Given the unavailability of objective, granular, and scalable individual-level SES measures in health care data sources, potential bias in artificial intelligence (“AI”) models by SES is under-studied and poorly understood, which includes the impact of SES on differential health care access and electronic health records (“EHRs”) quality. Thus, the HOUSES index (and individual-level SES) can be used for assessing, monitoring, and mitigating AI model bias by SES when AI models are applied to clinical care.

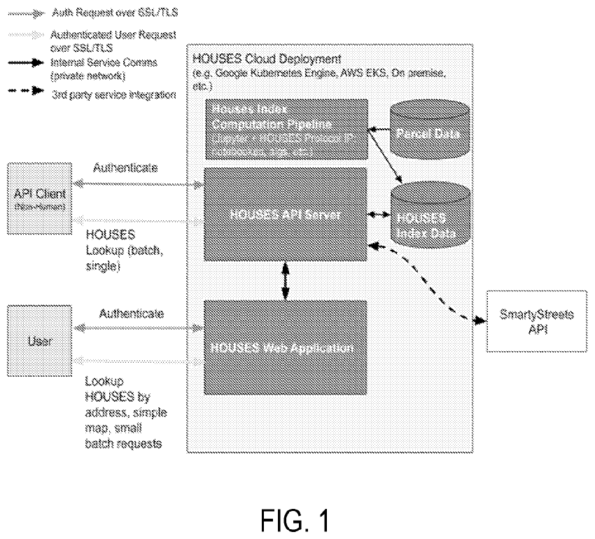

FIG. 1 shows a block diagram of an example HOUSES cloud system for generating and managing HOUSES index data. In general, a user authenticates and accesses a web application to perform a HOUSES index lookup via search and small batch uploads. Additionally or alternatively, an API client (e.g., electronic health record (“EHR”)) can authenticate directly to the API server to perform HOUSES index lookup requests. Secure, non-public HOUSES index intra-service and database communication is also enabled within the system. External third party integration with address lookups can also be provided.

At present, no individual-level SES measures of a population can be acquired via a scalable cloud-based and API-capable service. The HOUSES cloud system disclosed in the present disclosure enables automated downloading service of HOUSES index for a population by end users or subscribers anywhere and anytime. In general, the HOUSES cloud system includes at least the following three aspects: HOUSES index formulation, HOUSES index matching with addresses of users' dataset, and HOUSES index delivery or downloading.

HOUSES index formulation is the process of calculating HOUSES indices and metrics for housing parcels. An example of the calculation of a HOUSES index is described below in more detail. Additionally, formulating HOUSES index incorporated in software can address scenarios including algorithms for handling missing value, multi-unit housing or apartment complex, and mobile homes.

The HOUSES cloud system leverages cloud infrastructure and architectures to support: pipeline codification, scalability via distributed processing, repeatability and agility, and extensible imputation processing. For example, pipeline codification can include the pipeline of steps to import, clean, compute, and then store output HOUSES indices, which can be codified in re-runnable pipelines. Scalability via distributed processing can be implemented using distributed processing technologies, such as Apache Spark, to enable large scale data processing. Repeatability and agility can be realized as follows. By using the pipeline and scalability capabilities previously described, data cohorts can be re-run end-to-end, a subset of pipeline steps, and/or algorithm modifications to perform “what if” modeling. These steps can be facilitated using data science notebooks (e.g., Jupyter), for example, hosted within the HOUSES cloud system. Extensible imputation processing can be realized by using building classification, architectural codes, other parcel metrics, machine learning algorithms, clustering, state and nation-wide datasets, and the like. The systems and methods described in the present disclosure provide the ability to use heterogeneous algorithms/toolkits to provide best performing models.

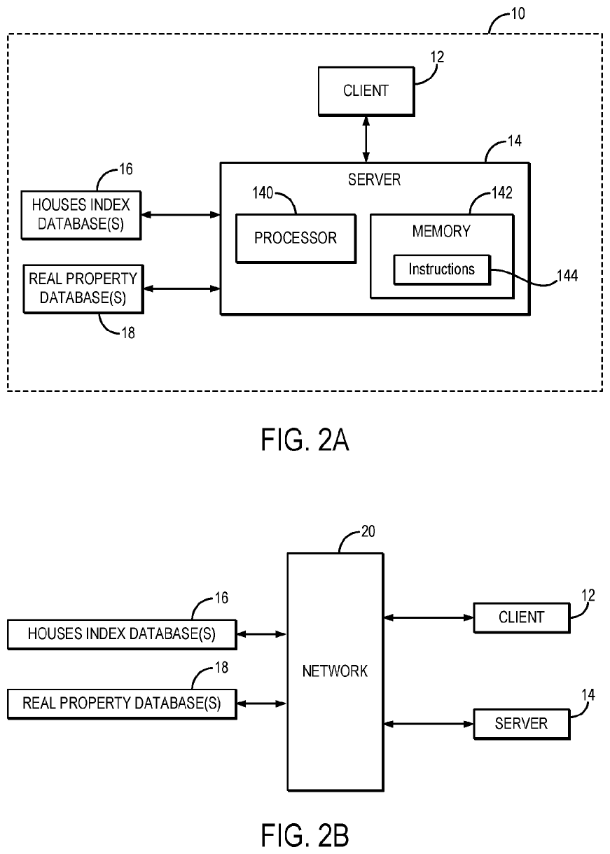

FIG. 2A illustrates an example housing-based socioeconomic status (“HOUSES”) index generation and management system 10. The system 10 includes a client 12 that communicates with a HOUSES index server 14 to order a HOUSES index formulation and/or lookup depending on the user and desired task. As noted above, the client 12 can include a computer system operated by a user, or can alternatively include an API client that can authenticate directly with the HOUSES index server 14. The HOUSES index server 14 is in communication with several databases, including one or more HOUSES index database(s) 16 and real property database(s) 18.

The client 12 can include a hardware processor, a memory, one or more inputs, and a display. In some examples, the client 12 can include a desktop computer, a laptop computer, a tablet device, a mobile device, or the like. Additionally or alternatively, the client 12 can include an API client. The client 12 communicates with the server 14, for example, to transmit address data for a HOUSES index formulation and/or lookup task, to receive HOUSES index data, or a combination thereof.

The client 12 generally provides a user interface through which a user can communicate requests to the HOUSES index server 14. The client 12 may, for example, generate a graphical user interface to facilitate requesting the formulation or retrieval of a HOUSES index score for an individual based on their relevant address information. For instance, a user can generate a HOUSES index request order for formulating and/or retrieving a HOUSES index based on an address for an individual, and this HOUSES index request order can be processed by the HOUSES index server 14 to query the respective database(s) and formulate and/or retrieve the respective data.

To this end, a HOUSES index request order can include address data input by the user at the client 12. Additionally or alternatively, the client 12 can include an API client that can authenticate directly with the HOUSES index server 14 to send a HOUSES index request order containing address data. Address data may include one or more of a street address (e.g., street number, street name, unit number as applicable), a municipality name (e.g., city name, village name, town name), a county name, a state name, a postal code (e.g., ZIP code, ZIP+4 code), a property tax key identifier, a parcel identifier, a Census tract identifier (e.g., a Census tract code, one or more Census block numbers), or the like.

The server 14 includes a server electronic control assembly having a server electronic processor 140 and a server memory 142. The server electronic processor 140 receives address data (e.g., via the client 12), stores the received address data in the server memory 142, and, in some embodiments, uses the address data for formulating and/or retrieving HOUSES index data. The server 14 may maintain the HOUSES index database(s) 16, the real property database(s) 18, or other databases (e.g., on the server memory 142), or these databases may be maintained as separate databases that are accessible by the server 14.

Although illustrated as a single device, the server 14 may be a distributed device in which the server electronic processor 140 and server memory 142 are distributed among two or more units that are communicatively coupled (e.g., via the network 20).

The server electronic processor 140 and the server memory 142 can communicate over one or more control buses, data buses, etc. The use of one or more control and/or data buses for the interconnection between and communication among the various modules, circuits, and components would be known to a person skilled in the art.

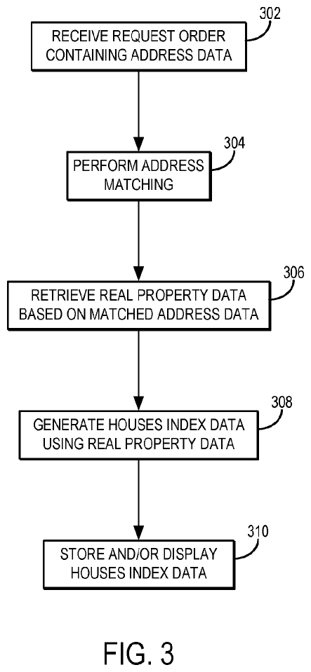

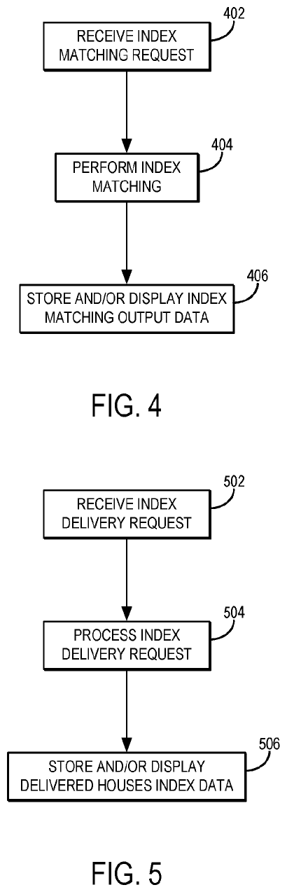

The server electronic processor 140 can be configured to communicate with the server memory 142 to store data and retrieve stored data. The server electronic processor 140 can be configured to receive instructions and data from the server memory 142 and execute, among other things, the instructions. In particular, the server electronic processor 140 executes instructions stored in the server memory 142. Thus, the server electronic controller coupled with the server electronic processor 140 and the server memory 142 can be configured to perform the methods described herein (e.g., the process 300 of FIG. 3, the process 400 of FIG. 4, and/or the process 500 of FIG. 5).

The server memory 142 can include read-only memory (“ROM”), random access memory (“RAM”), other non-transitory computer-readable media, or a combination thereof. The server memory 142 can include instructions 144 for the server electronic processor 140 to execute. The instructions 144 can include software executable by the server electronic processor 140 to enable the server electronic controller to, among other things, receive address data from the client 12, retrieve real property data associated with the address data from the real property database(s) 18, formulate a HOUSES index score based on the real property data, and send the HOUSES index score to the client 12 and/or store the HOUSES index score in the HOUSES index database(s) 16. Alternatively, the instructions 144 can include software executable by the server electronic processor 140 to enable the server electronic controller to, among other things, receive address data from the client 12 and retrieve a HOUSES index score from the HOUSES index database(s) 16 based on the address data. The software can include, for example, firmware, one or more applications (e.g., including web applications), program data, filters, rules, one or more program modules, and other executable instructions.

The server electronic processor 140 is configured to retrieve from server memory 142 and execute, among other things, instructions 144 related to the control processes and methods described herein. The server electronic processor 140 is also configured to store data on the server memory 142 including address data, HOUSES index data, real property data received from the real property database(s) 18, etc. Additionally or alternatively, the server electronic processor 140 is configured to store these data on the HOUSES index database(s) 16 and/or real property database(s) 18.

In these implementations, the HOUSES index server 14 can retrieve HOUSES index data from the HOUSES index database(s) 16 according to parameters (e.g., address data) submitted or otherwise queried by the user. In similar implementations, the HOUSES index server 14 can receive a HOUSES index request order from the client 12 to retrieve real property data from the real property database(s) 18 and to formulate a HOUSES index based on the retrieved real property data. In these implementations, the HOUSES index server 14 can retrieve the requested real property data (e.g., number of bedrooms, number of bathrooms, square footage of the unit, and estimated building value of the unit) from the real property database(s) 18 according to parameters (e.g., address data) submitted or otherwise queried by the user.

In general, the HOUSES index database(s) 16 store HOUSES index scores, or other such data associated with the HOUSES index scores (e.g., modified HOUSES index). The real property database(s) 18 store relevant real property data (e.g., number of bedrooms, number of bathrooms, square footage of the unit, and estimated building value of the unit) and in some embodiments may include real property data accessed from a county Assessor's office, or the like.

The HOUSES index database(s) 16 and/or real property database(s) 18 can be any suitable database for storing information such as HOUSES index scores, real property data (e.g., number of bedrooms, number of bathrooms, square footage of the unit, and estimated building value of the unit), and the like. In some examples, the HOUSES index database(s) 16 and/or real property database(s) 18 can implement a SQL database.

The network 20 may be a long-range wireless network such as the Internet, a local area network (“LAN”), a wide area network (“WAN”), or a combination thereof. In other embodiments, the network 20 may be a short-range wireless communication network, and in yet other embodiments, the network 20 may be a wired network. In some embodiments, the network 20 may include both wired and wireless devices and connections.

Although illustrated as a single network, the network 20 may include more than one network that separately connect various components of the HOUSES index generation and management system 10 together. For example, in some embodiments the client 12 and server 14 can be connected together via a first network, and the server 14 and the HOUSES index database(s) 16 and/or real property database(s) 18 can be connected together via a second network. In these instances, the first network may be a WAN while the second network may be a private network (e.g., a private LAN) that enables the server 14 and the HOUSES index database(s) 16 and/or real property database(s) 18 to communicate sensitive information therebetween using internal service communications or the like.

As shown in FIG. 2B, communication between the client 12, the HOUSES index server 14, and the databases (e.g., HOUSES index database(s) 16, real property database(s) 18) can be implemented via a communication network 20 that is configured to operate as a service layer or middleware.

The HOUSES index generation and management system 10 described here can implement a client application on the client 12 that works together with the HOUSES index server 14 and databases (e.g., HOUSES index database(s) 16, real property database(s) 18) to create, manage, and/or store HOUSES index scores and related information. As such, the described system 10 can securely store and make accessible HOUSES index scores via a cloud-based framework.

Users can launch an application at the client 12 (e.g., the client application) to both place a new HOUSES index request order and view any outstanding HOUSES index request orders. Additional views provided on the user interface of the client 12 can include a historical search for viewing and an ability to edit or cancel past work order entries stored in the worklist that are not in a completed state. Additionally or alternatively, the client 12 can be API client that makes requests via an API.

HIPAA compliance can be realized by encrypting all data at rest. HOUSES indices can be stored on encrypted file systems (e.g., encrypted cloud vendor storage). Temporary user provided inputs (e.g., request orders, address data) can also be stored on encrypted file systems. These temporary files can be used to service large bulk requests (e.g., bulk upload requests). As an example is an S3 bucket with encryption and no public access with whitelist access control list (“ACL”) to the server 14. Furthermore, no personal identifiable information (“PII”) is stored within the HOUSES index generation and management system 10. In some embodiments, the temporary files utilized to service large bulk requests may include some PII, but the temporary lifecycle of these files ensures that the PII is not retained in the HOUSES index generation and management system 10. Further still, API responses from the server 14 do not include PII.

All communications made by the server 14 are also protected. For example, all connectivity over the network 20 can be made using a transport layer security (“TLS”) protocol, or other similar secure communication protocol that encrypts communications between the end-user and/or client 12 to the server 14 and its cloud service APIs. As an example, service APIs to the server 14 require authentication, such as by using standard bearer token (e.g., Javascript Object Notation (“JSON”) web token (“JWT”), or the like) after successful authentication. Authentication, authorization, and API metrics can also be captured, logged, and stored by the server 14 (e.g., stored on the server memory 142 or on another memory, data storage device, or database).

Referring now to FIG. 3, a flowchart is shown illustrating the steps of an example process 300 for formulating a HOUSES index score based on address data provided by the client. The general flow of the HOUSES index formulation pipeline includes receiving address data, performing address matching, retrieving the relevant real property data, and generating a HOUSES index based on the real property data.

The method includes receiving a request order containing address data at the server 14, as indicated at step 302. For example, the address data can be received by the server 14 from the client 12. As one example, the client 12 can communicate the address data as an input received by a user, such as via a graphical user interface or other user interface. As another example, the client 12 can communicate the address data in response to a request received from the server 14, such as an API call or other request. As described above, the address data may include one or more of a street address (e.g., street number, street name, unit number as applicable), a municipality name (e.g., city name, village name, town name), a county name, a state name, a postal code (e.g., ZIP code, ZIP+4 code), a property tax key identifier, a parcel identifier, a Census tract identifier (e.g., a Census tract code, one or more Census block numbers), or the like. The request order may include address data for a single individual (i.e., a single housing unit address), or may be a batch request order or a bulk request order containing address data for multiple individuals and their respective housing unit addresses.

The received address data can then be matched using an address matching process, generating output as normalized address data, as indicated at step 304. For instance, the server processor 140 can perform address matching by matching the address data (e.g., each parcel) against external address data reference in order to normalize address components. As an example, the server processor 140 can request external address data from a third party data source using, for example, an address lookup API. Additionally or alternatively, the server processor 140 can retrieve external address data from the real property database(s) 18. Address matching can be persisted at different levels of granularity to support different address matching modalities (e.g., exact, parent address, street address, ZIP+4, ZIP code).

In some embodiments, address matching may include generating a composite address key and using the composite address key to perform the address matching. For example, a composite address can be created using address components computed during the address matching step. Composite addresses can be used to facilitate matching of end-user provided lookups.

The normalized address data are then used to retrieve real property data associated with the address data, as indicated at step 306. For example, the normalized address data can be used to query to real property database(s) 18 to retrieve relevant real property data. As described above, the real property data can include the number of bedrooms, number of bathrooms, square footage of the unit, and estimated building value of the unit. Additionally or alternatively, the real property data can include other data including ownership status, lot size of the housing unit, residential status (e.g., whether a housing unit is in a residential zoning and if so, which zoning district type), and the like. In some embodiments, additional real property data can be retrieved from sources other than the real property database(s) 18. For example, external real property data can be retrieved from a third party source and used to enrich or otherwise supplement the real property data. The external real property data may include, for example, census sourced data, apartment data, etc.

Using the real property data as input, a HOUSES index is generated by the server 14 (e.g., using the server processor 140), generating output as HOUSES index data, as indicated at step 308. In general, a HOUSES index can be computed as described above and by Y. J. Juhn, et al., in “Development and initial testing of a new socioeconomic status measure based on housing data,” J Urban Health, 2011:88 (5): 933-944, which is herein incorporated by reference in its entirety. For example, a HOUSES index score can be formulated by summing all variables of each real property data factor after transforming variables to z-scores. Alternatively, a HOUSES index score can be formulated by summing weighted variables using factor loadings on each real property data factor and comparing the results with z-score-based results. In some implementations, the HOUSES index can be computed while accounting for handling of missing values, multi-unit housing and/or apartment complexes, and mobile homes.

The output HOUSES index data are then stored by the server 14, as indicated at step 310. For example, the HOUSES index data may be stored in the HOUSES index database(s) 16, the server memory 142, or both. In some instances, additional data may also be stored together with the HOUSES index data, including related supplementary data stored as a geospatially indexed set of metrics.

The HOUSES index data may also be presented to a user, such as by communicating the HOUSES index data from the server 14 to the client 12 (e.g., via the network 20) and displaying or otherwise presenting the HOUSES index data to the user via the client 12 (e.g., via a display and/or graphical user interface).

In some embodiments, such as mentioned above, the HOUSES index data may be used to assess and mitigate AI model bias driven by a patient's SES. Given the significant associations of SES with health risk and health care access (especially driven by upstream social determinants of health (“SDH”)), quantifying the degree of bias in model performance by SES has important ethical implications for the use of AI in health care applications. Current AI fairness analyses are limited to considering readily available demographic factors such as age, sex, and race/ethnicity, leaving the role of SES in AI bias (on its own, or in interactions with other factors) poorly understood. Advantageously, the HOUSES index data generated by the systems and methods described in the present disclosure can be used to assess and mitigate AI model bias by individual-level SES.

To address this challenge in the equitable implementation of health care AI, the HOUSES index data can be used as a measure of SES with important features (e.g., validity, precision, objectivity (instead of self-report), and scalability) that can be integrated with AI model development. As a non-limiting example, differential data availability and quality of EHR data among study subjects according to SES as measured by HOUSES indices can be assessed, and HOUSES index data can be applied to quantify bias in commonly used metrics of model performance by SES.

Common metrics for assessing fairness in model performance can be used, such as accuracy equality (equal accuracy across groups), equal opportunity (equal sensitivity, 1 minus false negative rate [FNR] across groups), predictive equality (equal false positive rate [FPR] across groups), and predictive parity (equal precision across groups). Because it is impossible for a model to simultaneously satisfy all fairness metrics (e.g., equal opportunity, predictive equality, and predictive parity), and because there is currently no agreed-upon gold standard metric to be used, a balanced error rate (“BER”), which is defined as the unweighted average of the FPR (predictive equality) and FNR (equal opportunity), can advantageously be used as a metric for assessing bias (see Table 4 below for definitions of the metrics). BER can be advantageously chosen as a primary metric when the focus is on prediction accuracy, which involves both FPR (or 1-specificity) and FNR (or 1-sensitivity). The unweighted (i.e., equal weights) average can be used for summarizing both metrics, because the relative importance of these metrics will likely depend on the purpose of the studies.

For each desired metric, the ratio comparing least privileged group (e.g., HOUSES Q1 representing lower SES) with the privileged group (HOUSES Q2-Q4 representing higher SES) is computed. For FPR and BER, a ratio>1 means that the model performance is superior for the privileged group, while a ratio>1 for the other 3 metrics (accuracy equality, equal opportunity, and predictive parity) means the model performance is superior for the less privileged group. A ratio that is <0.8 or >1.25 (1/0.8) can be considered as indicating a meaningful difference, which is implemented in the open source program AI Fairness 360.

As a non-limiting example, in an example study, algorithmic bias for two different machine learning models (a Naïve Bayes (“NB”) model and a gradient boosting machine (“GBM”) model) for binary classification for estimating one-year asthma exacerbation (“AE”) risk among pediatric asthmatics were quantified by demographic factors (age, sex, race/ethnicity), SES (HOUSES and ADI), and chronic condition. To see the association of SES with data availability and completeness of EHR, the proportions of subjects with missing or unknown information for 7 variables relevant to asthma management were also calculated. This analysis can be done using HOUSES only, because the number of subjects with the lowest SES measured by ADI was very small. One variable was assessed as the main measure of data accuracy: diagnosed versus undiagnosed asthma by ICD codes for those who met predetermined asthma criteria (“PAC”). This calculation was done in both the training and testing cohorts.

The training cohort in this example included subjects with 71% being <12 years old and 57% males. For race/ethnicity, a large portion of subjects (60%) were non-Hispanic White and 14% were African American as shown in Table 1. Roughly 20% of the subjects were in the low-SES (HOUSES Quartile 1, Q1) group and 20% had at least one chronic condition. However, the proportions of subjects with lower SES by ADI were only 7% in training and 8% in testing cohorts. Subject characteristics were similar between training and testing cohorts. Roughly 30% of subjects had AE within one-year follow-up period (26% in the training cohort and 35% in the testing cohort: Table 3). Table 2 showed that proportion of AE differed by subject characteristics. In general, the proportion was higher in subjects who were younger, male, lower SES by HOUSES, and those with chronic conditions. There was significant discrepancy in the proportion of subjects with a history of AE among lower SES group defined by HOUSES (53%) and ADI (0%) in testing cohort.

TABLE 1
Subject characteristics used in the study

| | Training cohort | Testing cohort |
|---|---|---|
| | (N = 133) | (N = 113) |
| Age (in years), n (%) | | |
| <12 | 94 (71%) | 80 (71%) |
| ≥12 | 39 (29%) | 33 (29%) |
| Sex, n (%) | | |
| Male | 76 (57%) | 67 (59%) |
| Female | 57 (43%) | 46 (41%) |
| Race/ethnicity, n (%) | | |
| Non-Hispanic Whites | 76 (60%) | 67 (60%) |
| African Americans | 18 (14%) | 9 (8%) |
| Asians | 10 (8%) | 13 (12%) |
| Hispanics | 9 (7%) | 11 (10%) |
| Other categories | 14 (11%) | 12 (11%) |
| Missing | 6 | 1 |
| HOUSES, n (%) | | |
| Q1 (the lowest SES) | 22 (18%) | 15 (14%) |
| Q2-Q4 | 102 (82%) | 92 (86%) |
| Missing | 9 | 6 |
| Chronic condition, n (%) | | |
| Yes | 30 (23%) | 19 (17%) |
| No | 103 (77%) | 94 (83%) |
| National ADI, n (%) | | |
| 76-100 (the lowest SES) | 6 (7%) | 6 (8%) |
| 0-75 | 76 (93%) | 65 (92%) |
| Missing | 51 | 42 |
| Asthma exacerbation, n (%) | | |
| Yes | 34 (26%) | 40 (35%) |
| No | 99 (74%) | 73 (65%) |

TABLE 2
Proportion of subjects with asthma exacerbation (AE) by subject characteristics

| | Training cohort | | | | Testing cohort | | | |
|---|---|---|---|---|---|---|---|---|
| | (N = 133) | | | | (N = 113) | | | |
| | Subjects | | Subjects | | Subjects | | Subjects | |
| | with AE | | without AE | | with AE | | without AE | |
| | (N = 34) | | (N = 99) | | (N = 40) | | (N = 73) | |
| Age (in years), n (%) | | | | | | | | |
| <12 | 28 | (29.8%) | 66 | (70.2%) | 30 | (37.5%) | 50 | (62.5%) |
| ≥12 | 6 | (15.4%) | 33 | (84.6%) | 10 | (30.3%) | 23 | (69.7%) |
| Sex, n (%) | | | | | | | | |
| Male | 25 | (32.9%) | 51 | (67.1%) | 23 | (34.3%) | 44 | (65.7%) |
| Female | 9 | (15.8%) | 48 | (84.2%) | 17 | (39.1%) | 28 | (60.9%) |
| Race/ethnicity, n (%) | | | | | | | | |
| Non-Hispanic Whites | 19 | (25.0%) | 57 | (75.0%) | 25 | (37.3%) | 42 | (62.7%) |
| African Americans | 5 | (27.8%) | 13 | (72.2%) | 4 | (44.4%) | 5 | (55.6%) |
| Asians | 2 | (20.0%) | 8 | (80.0%) | 3 | (23.1%) | 10 | (76.9%) |
| Hispanics | 4 | (44.4%) | 5 | (55.6%) | 3 | (27.3%) | 8 | (72.7%) |
| Other categories | 4 | (28.6%) | 10 | (71.4%) | 4 | (33.3%) | 8 | (66.7%) |
| HOUSES, n (%) | | | | | | | | |
| Q1 (the lowest SES) | 6 | (27.3%) | 16 | (72.7%) | 8 | (53.3%) | 7 | (46.7%) |
| Q2-Q4 | 23 | (22.5%) | 79 | (77.5%) | 29 | (31.5%) | 63 | (68.5%) |
| Chronic condition, n (%) | | | | | | | | |
| Yes | 10 | (33.3%) | 20 | (66.7%) | 7 | (36.8%) | 12 | (63.2%) |
| No | 24 | (23.3%) | 79 | (76.7%) | 33 | (35.1%) | 61 | (64.9%) |
| National ADI, n (%) | | | | | | | | |
| 76-100 (the lowest SES) | 2 | (33.3%) | 4 | (66.7%) | 0 | (0.0%) | 6 | (100.0%) |
| 0-75 | 16 | (21.1%) | 60 | (78.9%) | 21 | (32.3%) | 44 | (67.7%) |

TABLE 3
Assessment of algorithmic bias for 2 machine learning models (Naïve Bayes [NB] and gradient boosting
machine [GBM]) estimating 1-year asthma exacerbation risk in childhood asthma using 5 commonly used bias metrics

| | | | | | | | | | Balanced error | |
|---|---|---|---|---|---|---|---|---|---|---|
| | Accuracy | | Equal opportunity | | Predictive | | Predictive | | rate | |
| | equality | | (sensitivity) | | parity (PPV) | | equality (FPR) | | ([FPR + FNR)/2] | |
| | NB | GBM | NB | GBM | NB | GBM | NB | GBM | NB | GBM |
| Groups | model | model | model | model | model | model | model | model | model | model |
| SES (HOUES) | | | | | | | | | | |
| Q1 (lowest SES) | 0.47 | 0.47 | 0.38 | 0.50 | 0.50 | 0.50 | 0.43 | 0.57 | 0.53 | 0.54 |
| Q2-Q4 | 0.62 | 0.50 | 0.59 | 0.76 | 0.43 | 0.36 | 0.37 | 0.62 | 0.39 | 0.43 |
| Ratio (Q1/Q2-4) (1 = no diff) | 0.75 | 0.93 | 0.64 | 0.66 | 1.18 | 1.39 | 1.17 | 0.92 | 1.35 | 1.25 |
| Age | | | | | | | | | | |
| <12 | 0.53 | 0.45 | 0.57 | 0.70 | 0.41 | 0.38 | 0.50 | 0.70 | 0.47 | 0.50 |
| ≥12 | 0.76 | 0.64 | 0.40 | 0.80 | 0.67 | 0.44 | 0.09 | 0.44 | 0.34 | 0.32 |
| Ratio (<12/≥12) (1 = diff) | 0.69 | 0.71 | 1.42 | 0.88 | 0.61 | 0.84 | 5.75 | 1.61 | 1.36 | 1.57 |
| Sex | | | | | | | | | | |
| Male | 0.49 | 0.45 | 0.48 | 0.78 | 0.33 | 0.36 | 0.50 | 0.73 | 0.51 | 0.47 |
| Female | 0.74 | 0.59 | 0.59 | 0.65 | 0.67 | 0.46 | 0.17 | 0.45 | 0.29 | 0.40 |
| Ratio (male/female) (1 = no diff) | 0.67 | 0.76 | 0.81 | 1.21 | 0.50 | 0.79 | 2.90 | 1.62 | 1.75 | 1.18 |
| Race/Ethnicity | | | | | | | | | | |
| Others | 0.54 | 0.39 | 0.47 | 0.60 | 0.35 | 0.29 | 0.42 | 0.71 | 0.48 | 0.56 |
| Non-Hispanic White | 0.63 | 0.58 | 0.56 | 0.80 | 0.50 | 0.47 | 0.33 | 0.55 | 0.39 | 0.37 |
| Ratio (others/White) (1 = no diff) | 0.87 | 0.67 | 0.83 | 0.75 | 0.70 | 0.62 | 1.26 | 1.30 | 1.23 | 1.48 |
| Chronic condition | | | | | | | | | | |
| At least one | 0.53 | 0.47 | 0.20 | 0.80 | 0.20 | 0.33 | 0.33 | 0.67 | 0.57 | 0.43 |
| None | 0.61 | 0.50 | 0.59 | 0.69 | 0.46 | 0.39 | 0.38 | 0.60 | 0.39 | 0.46 |
| Ratio (≥1/none) (1 = no diff) | 0.87 | 0.94 | 0.34 | 1.16 | 0.43 | 0.86 | 0.88 | 1.11 | 1.44 | 0.95 |
| ADI | | | | | | | | | | |
| 76-100 | 0.60 | 0.60 | NC | NC | 0.00 | 0.00 | 0.40 | 0.40 | NC | NC |
| 0-75 | 0.64 | 0.54 | 0.60 | 0.80 | 0.44 | 0.39 | 0.35 | 0.58 | 0.37 | 0.39 |
| Ratio (76-100/0-75) (1 = no diff) | 0.95 | 1.11 | NC | NC | 0.00 | 0.00 | 1.15 | 0.69 | NC | NC |

NC: not comparable.
Ratios either greater than 1.2 or less than 0.8 (ie, an absolute difference between the ratio and 1 being greater than 0.2) were bolded.

TABLE 4
Metrics used for assessing algorithmic fairness used in the example study

| | | | Comparison | |
|---|---|---|---|---|
| Base metrics | Definition | Meaning | metric | Interpretation |
| Accuracy | (TP + TN)/(TP + | The proportion of | Accuracy | Is the model more |
| | FP + TN + FN) | patients correctly | equality | accurate on one group |
| | | classified by the | | than another? |
| | | model (range: 0-1; | | Ratio = 1: fair |
| | | higher score means | | Ratio < 1: unfavorable |
| | | better performance). | | to unprivileged group |
| | | | | Ratio > 1: favorable |
| | | | | to unprivileged group |
| Sensitivity | TP/(TP + FN) = | The proportion of | Equal | Are future incidences |
| (recall, true | 1 − FNR | patients classified | opportunity | of asthma exacerbation |
| positive rate) | | as case by the model | | detected equally |
| | | among true cases | | between two groups? |
| | | (range: 0-1; higher | | (Or, equivalently, are |
| | | score means better | | future incidences of |
| | | performance) | | asthma exacerbation |
| | | | | missed equally |
| | | | | between two groups?) |
| | | | | Ratio = 1: fair |
| | | | | Ratio < 1: unfavorable |
| | | | | to unprivileged group |
| | | | | Ratio > 1: favorable |
| | | | | to unprivileged group |
| False | FP/(FP + TN) | The proportion of | Predictive | Do both groups share |
| positive rate | | patients falsely | equality | an equal burden of |
| | | classified as case | | unnecessary worry |
| | | among those who | | from false positives? |
| | | are not cases, which | | Ratio = 1: fair |
| | | is same as 1-specificity | | Ratio < 1: favorable to |
| | | (range: 0-1; higher | | unprivileged group |
| | | score means worse | | Ratio > 1: unfavorable |
| | | performance) | | to unprivileged group |
| Positive | TP/(TP + FP) | The proportion of | Predictive | Are predictions on |
| predictive | | true cases among | parity | both groups equally |
| value | | those classified as | | useful for clinicians, |
| (Precision) | | cases by the model | | or does one group have |
| | | (range: 0-1; higher | | a higher proportion of |
| | | score means better | | false positives among |
| | | performance) | | predicted positives? |
| | | | | Ratio = 1: fair |
| | | | | Ratio < 1: unfavorable |
| | | | | to unprivileged group |
| | | | | Ratio > 1: favorable |
| | | | | to unprivileged group |
| Unweighted | [FP/(FP + TN) + | Average between | Balanced | (Interpretable as an |
| average of | FN/(TP + FN)]/2 | FPR (predictive | error rate | average of equal |
| FPR and | | equality) and FNR | | opportunity and |
| FNR | | (1-sensitivity). | | predictive equality) |
| | | Range: 0-.5 (higher | | Ratio = 1: fair |
| | | score means worse | | Ratio < 1: favorable to |
| | | performance) | | unprivileged group |
| | | | | Ratio > 1: unfavorable |
| | | | | to unprivileged group |

TP: true positives;
FP: false positives;
TN: true negatives;
FN: false negatives;
FPR: false positive rate;
FNR: false negative rate

Using the testing cohort, Table 3 summarizes the results of bias in model performance for both NB and GBM models in estimating one-year AE risk. Overall, model performance was not independent of patient characteristics such as age, sex, and chronic diseases as expected. Also, the two models did not have systematically different patterns compared to one another in how their performance differed by these factors. Higher SES as measured by HOUSES index was greatly associated with superior model performance. Specifically, children in lower SES groups had higher BERs than those in the higher SES group in both ML models (ratio=1.35 for NB model and 1.25 for GBM model), which exceed those for race/ethnicity (1.23 and 1.04, respectively). This differential performance by SES was driven more by FNR (=1-sensitivity; ratio=1.51 by NB and 2.01 by GBM model) than FPR (1.18 by NB and 0.92 by GBM model). This was also true for the equal opportunity (i.e., sensitivity) metric. Children in the higher SES group had significantly higher sensitivity in the performance of both models, compared to those in the lower SES group, to a greater extent than the difference by other demographic factors. The bias analysis using ADI was limited due to the lack of children experiencing AE among those having the lowest SES measured by ADI in the testing cohort. For example, 2 of 5 metrics (equal opportunity and BER) used were not computable because the denominator was zero. Also, positive predictive value (“PPV”) for those with ADI>75 was zero because the numerator was zero.

These study results suggest that lower SES, as measured by the HOUSES index, is associated with worse predictive model performance. A possible mechanism for this bias in performance is incomplete and inaccurate EHR data, as AI models perform better with larger amounts of and more accurate data, and unavailability and inaccuracy were associated with lower SES. In turn, this suggests adopting AI models biased by SES systematically aggravates inequity, alongside greater health risk and lower health care access.

Referring now to FIG. 4, a flowchart is shown illustrating the steps of an example process 400 for retrieving HOUSES index data from a database (e.g., HOUSES index database(s) 16) based on an index matching request made by the client. Index matching is the process of consuming end-user provided address components, plus year(s), and returning matching HOUSES index data.

An index matching request is received by the server 14, as indicated at step 402. The index matching request can be received from the client 12, which may be initiated by a user, an API client, or the like. The index matching request may include address data and/or normalized address data.

Upon receipt of the index matching request, the server 14 processes the request (e.g., using the server processor 140) and performs an index matching process, as indicated at step 404. The index matching can use a similar address matching algorithm as used for input data in the index formulation process described above. An index matching algorithm is used to create composite address keys used to best find matching HOUSES index records. When no direct match is found, a series of imputation algorithms using the “next best” composite key can be used (e.g., parent address).

The results of the index matching are then stored by the server 14, as indicated at step 406. For example, the output data from the index matching may be stored in the HOUSES index database(s) 16, the server memory 142, or both. The index matching output data may also be presented to a user, such as by communicating the data from the server 14 to the client 12 (e.g., via the network 20) and displaying or otherwise presenting the data to the user via the client 12 (e.g., via a display and/or graphical user interface).

Referring now to FIG. 5, a flowchart is shown illustrating the steps of an example process 500 for delivering HOUSES index data from a database (e.g., HOUSES index database(s) 16).

An index delivery request is received by the server 14, as indicated at step 502. The index delivery request can be received from the client 12, which may be initiated by a user, an API client, or the like. Upon receipt of the index delivery request, the server 14 processes the request (e.g., using the server processor 140) and performs an index delivery process, as indicated at step 504. For example, the index delivery can retrieve HOUSES index data from the HOUSES index database(s) 16, or the like. The index delivery can be implemented, for example, as a set of secured APIs running on the server 14 in a HIPAA compliant manner.

Index delivery can be performed by secured API in a batch or bulk mode. Both batch and bulk mode APIs can provide user input per an index matching process (e.g., the index matching process 400 of FIG. 4). Batch mode can support up to thousands of inputs (e.g., 10,000 inputs), while bulk mode can provide a mechanism to upload much larger files. Bulk response APIs can provide a mechanism to check the status of a bulk operation as well as retrieval endpoint details to fetch final results.

The delivered HOUSES index data may stored by the server 14, as indicated at step 506. For example, the output of the index matching data may be stored in the HOUSES index database(s) 16, the server memory 142, or both. The delivered HOUSES index data may also be presented to a user, such as by communicating the data from the server 14 to the client 12 (e.g., via the network 20) and displaying or otherwise presenting the data to the user via the client 12 (e.g., via a display and/or graphical user interface).

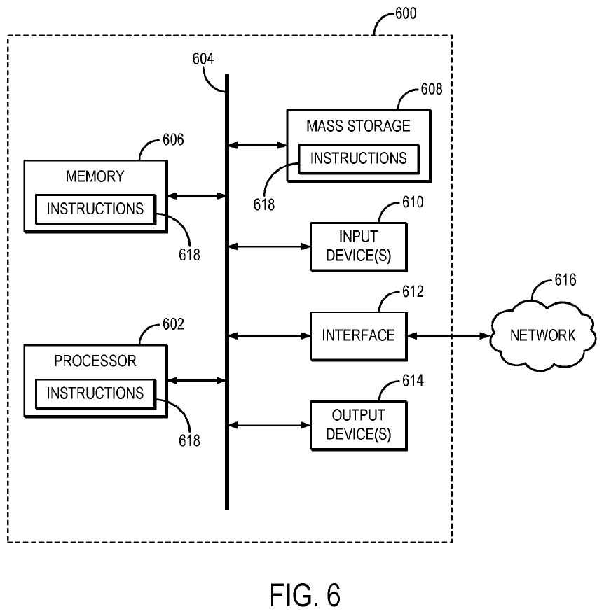

FIG. 6 is a block diagram illustrating an example of a computer system 600 that can implement systems, methods, and algorithms described here. The computer system 600 can include a processor 602 that is coupled to an interconnect 604, which may be an interconnection bus or the like. As an example, the processor 602 can be any suitable processor, processing unit, or microprocessor. Furthermore, the processor 602 may include a single processor or multiple different processors that are coupled to the interconnect 604.

The processor 602 is coupled to a memory 606 via the interconnect 604. The memory 606 can include any type of volatile memory, non-volatile memory, or combinations of both, including static random access memory (“SRAM”), dynamic random access memory (“DRAM”), flash memory, read-only memory (“ROM”), and so on.

The computer system 600 also includes a mass storage device 608, one or more input devices 610, an interface 612, and one or more output devices 614 that are connected to the interconnect 604. The one or more input devices 610 may include a keyboard, a mouse, a touch screen display, and so on. The interface 612 may be any suitable interface for wired or wireless communication between the computer system 600 and another computer system via a network 616. The one or more output devices 614 may include a display or the like.

The mass storage device 608 can include a machine-readable medium on which is stored one or more sets of data structures and instructions 618 (e.g., software) embodying or utilized by any one or more of the systems, methods, or algorithms described here. The instructions 618 may also reside, completely or at least partially, within the memory 606 or a local memory within the processor 602. The instructions 618 may also be transmitted or received over the network 616 and received by the computer system 600 via the interface 612.

In some embodiments, any suitable computer readable media can be used for storing instructions for performing the functions and/or processes described herein. For example, in some embodiments, computer readable media can be transitory or non-transitory. For example, non-transitory computer readable media can include media such as magnetic media (e.g., hard disks, floppy disks), optical media (e.g., compact discs, digital video discs, Blu-ray discs), semiconductor media (e.g., random access memory (“RAM”), flash memory, electrically programmable read only memory (“EPROM”), electrically erasable programmable read only memory (“EEPROM”)), any suitable media that is not fleeting or devoid of any semblance of permanence during transmission, and/or any suitable tangible media. As another example, transitory computer readable media can include signals on networks, in wires, conductors, optical fibers, circuits, or any suitable media that is fleeting and devoid of any semblance of permanence during transmission, and/or any suitable intangible media.

The present disclosure has described one or more preferred embodiments, and it should be appreciated that many equivalents, alternatives, variations, and modifications, aside from those expressly stated, are possible and within the scope of the invention.

1. A method for generating housing-based socioeconomic status (HOUSES) index scores for an individual, the method comprising:
(a) receiving a request order at a server by a client, wherein the request order comprises address data for an individual including a housing unit address for the individual;
(b) retrieving real property data from a real property database using the server and the address data to query the real property database, wherein the real property data comprise at least number of bedrooms, number of bathrooms, square footage of the unit, and estimated building value of the unit;
(c) generating HOUSES index scores with the server based on the real property data; and
(d) storing the HOUSES index scores on the server.

2. The method of claim 1, wherein storing the HOUSES index scores on the server includes storing the HOUSES index scores in a database.

3. The method of claim 1, wherein storing the HOUSES index scores on the server includes storing the HOUSES index scores in a memory of the server.

4. The method of claim 1, further comprising presenting the HOUSES index scores to a user.

5. The method of claim 4, wherein the HOUSES index scores are presented to the user via the client.

6. The method of claim 5, wherein the HOUSES index scores are transmitted to the client using an encrypted communication protocol.

7. The method of claim 1, wherein the request order is a user-initiated request order generated by the client in response to a user input.

8. The method of claim 1, wherein the request order is an application programming interface (API) initiated request order generated by the client.

9. The method of claim 8, wherein the request order includes an authentication request that is processed by the server to authenticate the client.

10. The method of claim 9, wherein the authentication request includes a bearer token.

11. The method of claim 10, wherein the bearer token comprises a JavaScript object notation web token (JWT).

12. The method of claim 1, further comprising generating normalized address data with the server by performing an address match of the address data in the request order in order to determine normalized address components for the address data and to generate normalized address data therefrom.

13. The method of claim 12, wherein the address match is persisted at different levels of granularity to support different address matching modalities.

14. The method of claim 12, wherein generating the normalized address data includes generating a composite address key from the address data and performing the address match using the composite address key.

15. The method of claim 1, wherein the server processes the request order and generates the HOUSES index scores without persisting any personal identifiable information of the individual.

16. The method of claim 1, wherein the real property data are retrieved from the real property database comprising county assessor data.

17. The method of claim 1, wherein the request order is received from the client using an encrypted communication protocol.

18. A method for quantifying artificial intelligence (AI) model bias by an individual-level socioeconomic status (SES), the method comprising:
accessing with a computer system, housing-based socioeconomic status (HOUSES) index scores for individuals in a study cohort, wherein the HOUSES index scores are generated based on real property data comprising at least number of bedrooms, number of bathrooms, square footage of a housing unit for each individual, and estimated building value of each housing unit;
computing a fairness metric based on the HOUSES index scores using the computer system; and
quantifying AI model bias by SES in the study cohort based on the fairness metric.

19. The method of claim 18, wherein the fairness metric comprises a balanced error rate metric.

20. The method of claim 19, wherein the balanced error rate metric is computed as a ratio comparing a least privileged group of individuals in the study cohort with a privileged group of individuals in the study testing cohort, wherein the least privileged group of individuals and the privileged group of individuals are selected based on the HOUSES index scores for the individuals in the study cohort.
